(19) United States
(12) Patent Application Publication
(10) Pub. No.: US 2013/0137946 A1
(43) Pub. Date: May 30, 2013

(54) MEDICAL DEVICE WITH CONDITIONAL POWER CONSUMPTION

(71) Applicants: Thomas Geske, Erie, CO (US); Nick Robertson, Erie, CO (US); Thomas Price, Denver, CO (US); Kalpathy Krishnan, Boulder, CO (US); James C. Gibson, Greeley, CO (US)

(72) Inventors: Thomas Geske, Erie, CO (US); Nick Robertson, Erie, CO (US); Thomas Price, Denver, CO (US); Kalpathy Krishnan, Boulder, CO (US); James C. Gibson, Greeley, CO (US)

(73) Assignee: Nellcor Puritan Bennett LLC, Boulder, CO (US)

(21) Appl. No.: 13/307,611

(22) Filed: Nov. 30, 2011

(51) Int. Cl. A61B 5/0205; A61B 5/145

(52) U.S. Cl. 600/324; 600/323

(57) ABSTRACT

Embodiments of the present disclosure relate to a system and method for reducing power consumption of a medical device based on one or more physiological parameters. For example, the medical device may be operated in a low power mode if a physiological parameter trend is above a certain threshold. In the low power mode, the processing power may be reduced relative to a high power mode. The low power mode may be associated with reduced processing and output rate.

### BACKGROUND

The present disclosure relates generally to techniques for monitoring physiological parameters of a patient. Specifically, embodiments of the present disclosure relate to reducing overall power consumption of a medical device by conditionally reducing processing power based on measurements of a physiological parameter.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring certain physiological characteristics of a patient. Such devices provide doctors and other healthcare personnel with the information they need to provide the best possible healthcare for their patients. As a result, such monitoring devices have become an indispensable part of modern medicine. For example, photoplethysmography is a common technique for monitoring physiological characteristics of a patient, and one device based upon photoplethysmography techniques is typically referred to as a pulse oximeter. Pulse oximeters may be used to measure and monitor various blood flow characteristics of a patient. A pulse oximeter may be utilized to monitor the blood oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time-varying amount of arterial blood in the tissue during each cardiac cycle.

A patient in a hospital setting may be monitored by a variety of medical devices, including devices based on pulse oximetry techniques. For example, a standalone pulse oximetry monitor may include a processor for receiving a sensor signal and using the signal to determine blood oxygen saturation. Depending on the configuration of the device, a power source coupled to the monitor may provide power to any associated sensors (e.g., one or more light sources and corresponding detectors), mechanical systems, a display, and the processors. For devices that are capable of using battery power, it is desirable to reduce power consumption to extend battery life. However, for devices that use a wall outlet as a power source, it may also be advantageous to include features that reduce power consumption to more efficiently distribute power within the device.

### BRIEF DESCRIPTION OF THE DRAWINGS

Advantages of the disclosed techniques may become apparent upon reading the following detailed description and upon reference to the drawings in which:

FIG. 1 is an illustration of a patient monitoring system in accordance with an embodiment;

FIG. 2 is a block diagram of a patient monitor that may operate in high power or low power modes in accordance with an embodiment;

FIG. 3 is a flow diagram of a method for determining a power mode in accordance with an embodiment;

FIG. 4 is an example of a trend of a physiological parameter that may be associated with a low power mode;

FIG. 5 is an example of a trend of a physiological parameter that may be associated with a high power mode; and

FIG. 6 is a flow diagram of a method for determining a power mode using alarm limit calculations in accordance with an embodiment.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Provided herein are techniques for reducing the power consumption of a medical device, e.g., a pulse oximeter. As discussed in greater detail below, the disclosure describes methods for obtaining a plethysmographic signal from a sensor coupled to a medical device and determining one or more physiological parameters from the signal. Based on one or more characteristics of the determined physiological parameter, the device may switch between a high power mode of operation and a low power mode of operation. For example, if a trend of the physiological parameter is relatively stable and associated with low clinical complications, a device may enter a low power mode. However, if the trend is unstable and/or if the measured value of the physiological parameter is changing relatively quickly, the device may enter a higher power mode. The high power mode and the low power mode may be characterized by changes in power allocation to one or more hardware components of the device. For example, the power provided to a light source of a patient sensor may be reduced in a low power mode, which in certain embodiments may also be associated with a reduced sampling rate for the detected signal. In one embodiment, in contrast to techniques in which the high power modes and low power modes are characterized by changes in hardware power consumption (e.g., a change in driving power to a light source), embodiments of the present disclosure also relate to changes in power distribution that are achieved via software, e.g., a change in processing power consumption.

In particular embodiments, a high power mode may be characterized by more frequent algorithm calculation or a higher refresh rate to determine the physiological parameter. That is, the high power mode may be associated with greater processing power. The high power mode may be appropriate for a patient with a rapidly-changing or highly variable physiological parameter. For such patients, more frequent calculation of the parameter may alert a caregiver to sudden changes in clinical condition. In contrast, patients with a relatively stable measured physiological parameter (e.g., with a flat trend line) may be adequately monitored with less frequent calculation of the parameter. This less frequent calculation may be characteristic of a low power mode, which may reduce the overall processing power of the device. Depending on the particular implementation, the determination of switching between the high power mode and the low power mode may be conditional and based on a trend line, a variability, a threshold, or a combination thereof for one or more physiological parameters.

While power consumption may be of concern for battery-powered medical devices, minimizing power consumption may also provide benefits for devices that operate using an outlet or a stationary power supply. The power consumption of a medical device may include any power used by associated sensors, mechanical systems such as cooling systems, a display, and the processors. Although the processing power may account for only a subset of the total power consumed by the medical device, more efficient processing may lead to lower average power consumption. This is turn may result in lower overall heating of the device during operation, which may result in less power consumed by cooling and exhaust systems. In another example, a multi-parameter monitoring system may include one or more modules that are capable of receiving photoplethysmography signals from a medical sensor and determining physiological parameters from the signal. Such multi-parameter systems may be capable of accepting a number of additional modules, each specific to a particular sensor or monitoring technique (e.g., pulse oximetry, ECG, blood pressure). The configuration of such multi-parameter systems may be limited by total power requirements of the system running with a complete set of associated modules. If one or more modules are configured to operate more efficiently via conditional processing reduction in a lower power mode, the multi-parameter system may be implemented with more modules or may be configured with smaller fans to account for more efficient power distribution. Alternatively, if the average power consumption of one module is reduced, the lower average may allow a more computationally complex module with higher average power consumption to be incorporated in the multi-parameter system or may allow processing power of one module to be redirected to another module. The techniques provided herein may be used in conjunction with a standalone monitor or with a multi-parameter monitoring device.

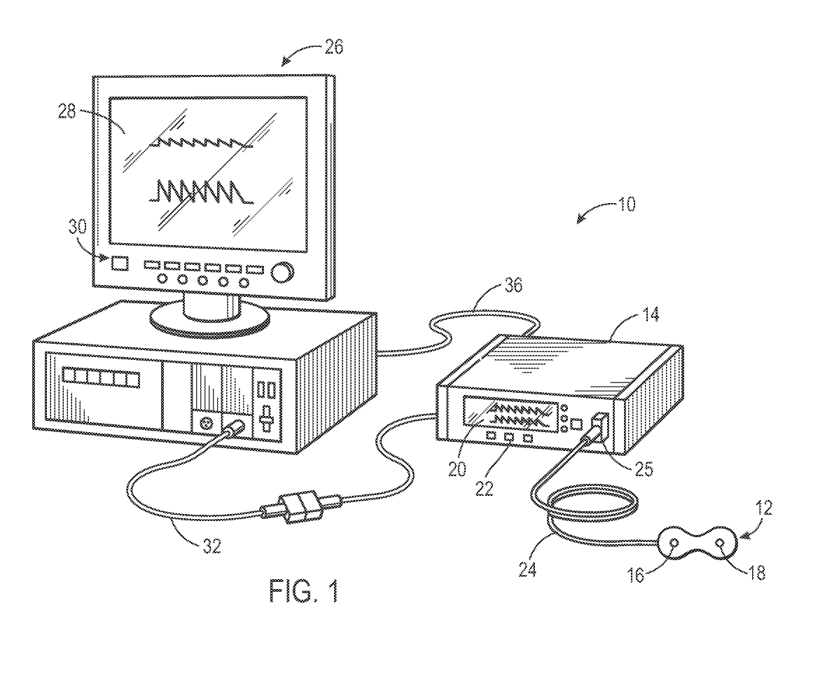

With this in mind, FIG. 1 depicts an embodiment of a patient monitoring system 10 that may be used in conjunction with a medical sensor 12. Although the depicted embodiments relate to photoplethysmography or pulse oximetry, the system 10 may be configured to obtain a variety of medical measurements with a suitable medical sensor. For example, the system 10 may, additionally be configured to determine tissue hydration, total hemoglobin, regional saturation, or any other suitable physiological parameter. As noted, the system 10 includes the sensor 12 that is communicatively coupled to a patient monitor 14. The sensor 12 includes one or more emitters 16 and one or more detectors 18. The emitters 16 and detectors 18 of the sensor 12 are coupled to the monitor 14 via a cable 24 through a plug 25 coupled to a sensor port. Additionally, the monitor 14 includes a monitor display 20 configured to display information regarding the physiological parameters, information about the system, and/or alarm indications. The monitor 14 may include various input components 22, such as knobs, switches, keys and keypads, buttons, etc., to provide for operation and configuration of the monitor. The monitor 14 also includes a processor that may be used to execute code such as code for implementing the techniques discussed herein.

The monitor 14 may be any suitable monitor, such as a pulse oximetry monitor available from Nellcor Puritan Bennett LLC. Furthermore, to upgrade conventional operation provided by the monitor 14 to provide additional functions, the monitor 14 may be coupled to a multi-parameter patient monitor 26 via a cable 32 connected to a sensor input port or via a cable 36 connected to a digital communication port, or via an RF or optical wireless link. Alternatively, the techniques provided herein may be incorporated into one or more individual modules with plug-in connectivity to the multi-parameter patient monitor 26. Such modules may include connectors that allow the calculated physiological parameters to be sent to the host multi-parameter monitor. In addition, the monitor 14, or, alternatively, the multi-parameter patient monitor 26, may be configured to calculate physiological parameters and to provide a central display 28 for the visualization of information from the monitor 14 and from other medical monitoring devices or systems. The multi-parameter monitor 26 includes a processor that may be configured to execute code. The multi-parameter monitor 26 may also include various input components 30, such as knobs, switches, keys and keypads, buttons, etc., to provide for operation and configuration of the a multi-parameter monitor 26. In addition, the monitor 14 and/or the multi-parameter monitor 26 may be connected to a network to enable the sharing of information with servers or other workstations. In certain embodiments, the sensor 12 may be a wireless sensor 12. Accordingly, the wireless sensor 12 may establish a wireless communication with the patient monitor 14 and/or the multi-parameter patient monitor 26 using any suitable wireless standard. By way of example, the wireless module may be capable of communicating using one or more of the ZigBee standard, WirelessHART standard, Bluetooth standard, IEEE 802.11x standards, or MiWi standard. In embodiments in which the sensor 12 is configured for wireless communication, the strain relief features of the cable 24 may be housed in the sensor body 34.

As provided herein, the sensor 12 may be a sensor suitable for detection of one or more physiological parameters. The sensor 12 may include optical components (e.g., one or more emitters 16 and detectors 18). In one embodiment, the sensor 12 may be configured for photo-electric detection of blood and tissue constituents. For example, the sensor 12 may include pulse oximetry sensing functionality for determining the oxygen saturation of blood as well as other parameters from the plethysmographic waveform detected by the oximetry technique. An oximetry system may include a light sensor (e.g., sensor 12) that is placed at a site on a patient, typically a fingertip, toe, forehead or earlobe, or in the case of a neonate, across a foot. The sensor 12 may pass light using the emitter 16 through blood perfused tissue and photoelectrically sense the absorption of light in the tissue. For example, the monitor 14 may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. The light intensity or the amount of light absorbed may then be used to calculate the amount of the blood constituent (e.g., oxyhemoglobin) being measured and other physiological parameters such as the pulse rate and when each individual pulse occurs. Generally, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. At least two, e.g., red and infrared (IR), wavelengths may be used because it has been observed that highly oxygenated blood will absorb relatively less red light and more infrared light than blood with a lower oxygen saturation. However, it should be understood that any appropriate wavelengths, e.g., green, etc., may be used as appropriate. Further, photoplethysmography measurements may be determined based on only one, two, or three or more wavelengths of light.

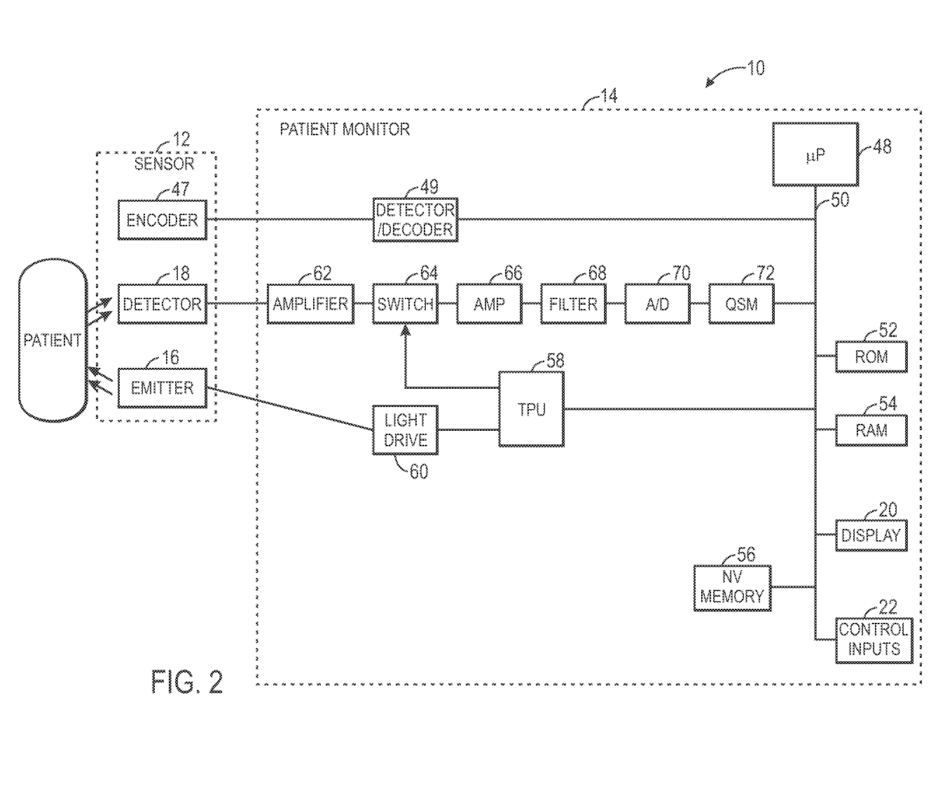

Turning to FIG. 2, a simplified block diagram of the medical system 10 is illustrated in accordance with an embodiment. As noted, the sensor 12 may include optical components in the forms of emitters 16 and detectors 18. The emitter 16 and the detector 18 may be arranged in a reflectance or transmission-type configuration with respect to one another. However, in embodiments in which the sensor 12 is configured for use on a patient's forehead (e.g. either alone or in conjunction with a hat or headband), the emitters 16 and detectors 18 may be in a reflectance configuration. Such sensors 12 may be used for pulse oximetry or regional saturation monitoring (e.g., INVOS® monitoring). An emitter 16 may also be a light emitting diode, superluminescent light emitting diode, a laser diode or a vertical cavity surface emitting laser (VCSEL). An emitter 16 and detector 18 may also include optical fiber sensing elements. An emitter 16 may include a broadband or “white light” source, in which case the detector could include any of a variety of elements for selecting specific wavelengths, such as reflective or refractive elements, absorptive filters, dielectric stack filters, or interferometers. These kinds of emitters and/or detectors would typically be coupled to the sensor 12 via fiber optics. Alternatively, a sensor assembly 12 may sense light detected from the tissue is at a different wavelength from the light emitted into the tissue. Such sensors may be adapted to sense fluorescence, phosphorescence, Raman scattering, Rayleigh scattering and multi-photon events or photoacoustic effects in conjunction with the appropriate sensing elements.

In certain embodiments, the emitter 16 and detector 18 may be configured for pulse oximetry. It should be noted that the emitter 16 may be capable of emitting at least two wavelengths of light, e.g., red and infrared (IR) light, into the tissue of a patient, where the red wavelength may be between about 600 nanometers (nm) and about 700 nm, and the IR wavelength may be between about 800 nm and about 1000 nm. The emitter 16 may include a single emitting device, for example, with two LEDs, or the emitter 16 may include a plurality of emitting devices with, for example, multiple LEDs at various locations. In some embodiments, the LEDs of the emitter 16 may emit three or more different wavelengths of light. Such wavelengths may include a red wavelength of between approximately 620-700 nm (e.g., 660 nm), a far red wavelength of between approximately 690-770 nm (e.g., 730 nm), and an infrared wavelength of between approximately 860-940 nm (e.g., 900 nm). Other wavelengths may include, for example, wavelengths of between approximately 500-600 nm and/or 1000-1100 nm and/or 1200-1400 nm. Regardless of the number of emitting devices, light from the emitter 16 may be used to measure, for example, oxygen saturation, water fractions, hematocrit, or other physiologic parameters of the patient. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure. In another embodiment, two emitters 16 may be configured for use in a regional saturation technique. To that end, the emitters 16 may include two light emitting diodes (LEDs) that are capable of emitting at least two wavelengths of light, e.g., red or near infrared light. In one embodiment, the LEDs emit light in the range of 600 nanometers to approximately 1000 nm. In a particular embodiment, one LED is capable of emitting light at 730 nm and the other LED is capable of emitting light at 810 nm.

In any suitable configuration of the sensor 12, the detector 18 may be an array of detector elements that may be capable of detecting light at various intensities and wavelengths. In one embodiment, light enters the detector 18 after passing through the tissue of the patient. In another embodiment, light emitted from the emitter 16 may be reflected by elements in the patient's tissue to enter the detector 18. The detector 18 may convert the received light at a given intensity, which may be directly related to the absorbance and/or reflectance of light in the tissue of the patient, into an electrical signal. That is, when more light at a certain wavelength is absorbed, less light of that wavelength is typically received from the tissue by the detector 18, and when more light at a certain wavelength is reflected, more light of that wavelength is typically received from the tissue by the detector 18. The detector 18 may receive light that has not entered the tissue to be used as a reference signal. After converting the received light to an electrical signal, the detector 18 may send the signal to the monitor 14, where physiological characteristics may be calculated based at least in part on the absorption and/or reflection of light by the tissue of the patient.

In certain embodiments, the medical sensor 12 may also include an encoder 47 that may provide signals indicative of the wavelength of one or more light sources of the emitter 16, which may allow for selection of appropriate calibration coefficients for calculating a physical parameter such as blood oxygen saturation. The encoder 47 may, for instance, be a coded resistor, EEPROM or other coding devices (such as a capacitor, inductor, PROM, RFID, parallel resident currents, or a colorimetric indicator) that may provide a signal to a microprocessor 48 related to the characteristics of the medical sensor 12 to enable the microprocessor 48 to determine the appropriate calibration characteristics of the medical sensor 12. Further, the encoder 47 may include encryption coding that prevents a disposable part of the medical sensor 12 from being recognized by a microprocessor 48 unable to decode the encryption. For example, a detector/decoder 49 may translate information from the encoder 47 before it can be properly handled by the processor 48. In some embodiments, the encoder 47 and/or the detector/decoder 48 may not be present. In some embodiments, the encrypted information held by the encoder 47 may itself be transmitted via an encrypted data protocol to the detector/decoder 49, such that the communication between 47 and 49 is secured.

Signals from the detector 18 and/or the encoder 47 may be transmitted to the monitor 14. The monitor 14 may include one or more processors 48 coupled to an internal bus 50. Also connected to the bus may be a ROM memory 52, a RAM memory 54, non-volatile memory 56, a display 20, and control inputs 22. A time processing unit (TPU) 58 may provide timing control signals to light drive circuitry 60, which controls when the emitter 16 is activated, and if multiple light sources are used, the multiplexed timing for the different light sources. TPU 58 may also control the gating-in of signals from detector 18 through a switching circuit 64. These signals are sampled at the proper time, depending at least in part upon which of multiple light sources is activated, if multiple light sources are used. The received signal from the detector 18 may be passed through one or more amplifiers (e.g., amplifiers 62 and 66), a low pass filter 68, and an analog-to-digital converter 70 for amplifying, filtering, and digitizing the electrical signals from the sensor 12. The digital data may then be stored in a queued serial module (QSM) 72, for later downloading to RAM 54 as QSM 72 fills up. In an embodiment, there may be multiple parallel paths for separate amplifiers, filters, and A/D converters for multiple light wavelengths or spectra received.

Based at least in part upon the received signals corresponding to the light received by optical components of the pulse oximetry sensor 20, microprocessor 48 may calculate the oxygen saturation and/or heart rate using various algorithms, such as those employed by the Nellcor™ N-600x™ pulse oximetry monitor, which may be used in conjunction with various Nellcor™ pulse oximetry sensors, such as OxiMax™ sensors. In addition, the microprocessor 48 may calculate and/or display trend or parameter variability using various methods, such as those provided herein. These algorithms may employ certain coefficients, which may be empirically determined, and may correspond to the wavelengths of light used. The algorithms and coefficients may be stored in a ROM 52 or other suitable computer-readable storage medium and accessed and operated according to microprocessor 48 instructions. In one embodiment, the correction coefficients may be provided as a lookup table.

In certain embodiments, the system 10 is capable of switching between a high power mode and a low power mode. A monitor 14 capable of switching between the high power mode and the low power mode using parameter conditions as an input may result in reduced overall average power consumption. In one example, pulse oximetry functionality for a monitor 14 or module may be below 100 mW or below 80 mW when the present techniques are implemented. During a high power mode, the processing power may account for greater than 30 mW of power consumption. A reduction in processing power to bring average power consumption of the processors to below 30 mW (or overall consumption below 80 mW) may achieve desired low power characteristics with or without additional power reduction through other techniques. However, the techniques provided herein may be used in combination with hardware-based techniques that achieve even further reductions in power consumption. For example, a monitor 14 may dissipate about 50 mW of power to the optical components of the sensor 12 (e.g., LED drive). In certain embodiments, one embodiment of a low power mode may reduce the power to the emitter 16. Other techniques for reducing power consumption may include dimming a display or reducing a signal sampling rate. Accordingly, a low power mode may include software-based and, in certain embodiments, hardware-based techniques for reducing power consumption.

The reduction in processing power may be achieved by changing a calculation rate for a physiological parameter. A high power mode may be associated with a more frequent calculation rate/second than a low power mode. In one embodiment, the calculation rate for a blood oxygen saturation and/or heart rate may be about 78 times/second, while a low power mode may be associated with a calculation rate of 50 times/second or less. In one embodiment, the calculation rate to about once per second may achieve adequate monitoring for a stable patient. Similarly, other parameters may have a characteristic calculation rate in a high power mode that is more frequent relative to a low power mode based on the appropriate physiological calculations for that parameter. For example, a respiration rate may be calculated about once every five seconds in a high power mode and once every 30 seconds in a low power mode. In addition, depending on the other power conditions for the monitor, the reduction in calculation frequency may be accompanied by a reduction in the sampling rate of the received signal. In configurations in which the drive cycle of the emitter is reduced to conserve power, the sampling rate is also reduced.

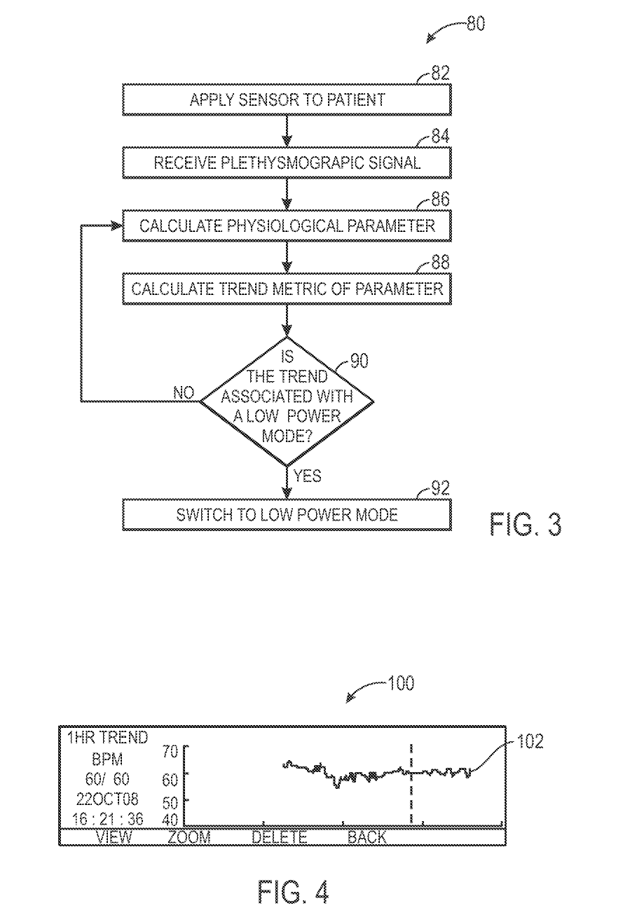

FIG. 3 is a process flow diagram illustrating a method 80 for selecting a power mode for a pulse oximetry monitor in accordance with certain embodiments. It should be understood that there may be, within the categories of high power and low power, various gradations of such modes, and that the system 10 is capable of automatically determining an appropriate power mode based on patient parameter conditions. The method may be performed as an automated procedure by a system, such as a system that includes a patient monitor 14 and a sensor 12. In addition, certain steps of the method may be performed by a processor, or a processor-based device such as a patient monitor 14 that includes instructions for implementing certain steps of the method 80. According to an embodiment, the method 80 begins with coupling a pulse oximetry sensor 12 to a patient at step 82 and receiving a pulse signal from the sensor 12 at step 84.

At step 86, the monitor 14 uses data from the sensor 12 over a period of time to calculate a physiological parameter such as blood oxygen saturation. In the depicted embodiment, the default setting of the monitor 14 is a high power mode. However, other implementations may include user-selected power modes. At step 88, a metric associated with a trend of the parameter is calculated based on individual data values. Trend metrics may include an instantaneous slope, a slope of the parameter over a predetermined time period, an absolute value of a slope, or a metric that combines slope data with average parameter data. Based on a trend metric of the blood oxygen saturation, the monitor 14 determines whether a low power mode is appropriate at step 90. If the low power mode is appropriate, the method 80 switches to a low power mode at step 92 by altering certain processing steps and reducing overall power consumption by processors (e.g., microprocessor 48). If the high power mode is appropriate, the method 80 returns to step 86. The method 80 may also include a display or indication of the power mode in use. While the monitor 14 may be implemented with a default high power mode and automatic switching to low power modes under appropriate conditions, the power mode selection may be based on user input and operate according to a truth table. For example, the monitor 14 may determine if a user selection of a low power mode is appropriate based on the physiological parameter trend.

As provided, the determination of the switch to low power mode may be based on one or more characteristics of trend data of a physiological parameter. For example, FIG. 4 is an example of a trend plot 100 of a heart rate, shown as beats per minute on line 102. In the depicted example, the trend line 102 is generally stable and is in a normal range around 60 bpm. For such a patient, a lower power mode may be appropriate because the patient's physiological condition is not changing. The time window for the determination of the trend may be selected by the caregiver or may be automatically determined. In the depicted example, the time window is about one hour. Depending on the time window selected, more weight may be given to more recent calculations relative to more distant calculations.

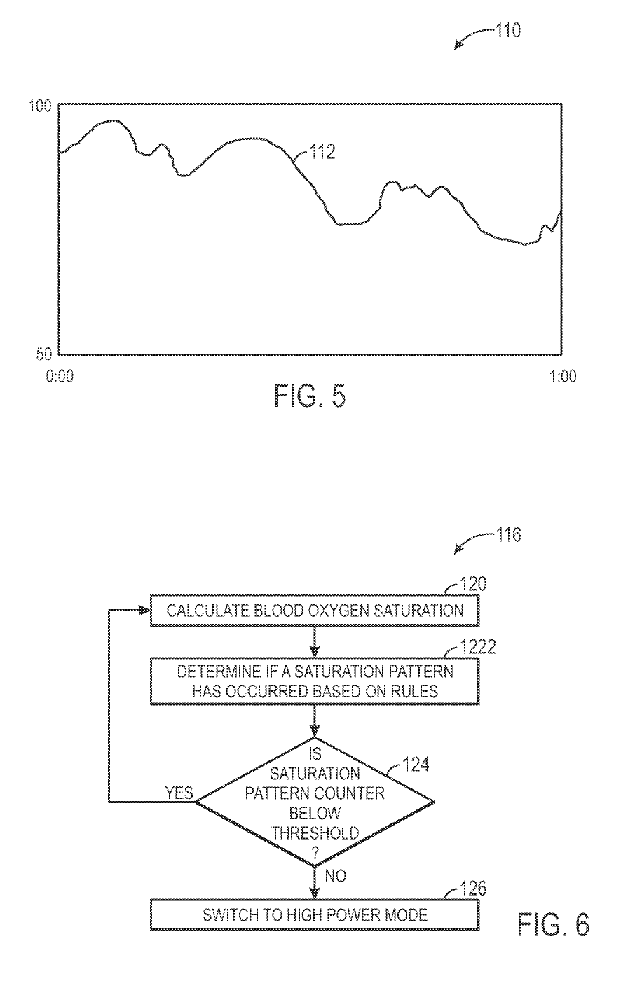

FIG. 5 is an example of a trend plot 110 of blood oxygen saturation shown as line 112 that is trending downward. In the depicted example, the downward trend may be associated with a high power mode. That is, the patient's condition may be sufficiently variable that the calculation rate of the parameter is increased. By switching to more frequent parameter calculation, variation or sudden changes may be assessed more accurately. This may be appropriate for a patient with a relatively variable or changing condition. In certain embodiments, the downward trend may be combined with a threshold to determine a change to the low power mode or to the high power mode. If downward trend is associated with values outside of normal, then the high power mode may be triggered more quickly than if the downward trend maintains the parameter within normal values.

In one embodiment, the trend of a particular physiological parameter may be quantified based on the rate of change for a given parameter and the switch between high power and low power modes may be based on the trend value. For example, a trend value outside of a predetermined limit or range may be associated with a high power mode. The trend may be an instantaneous slope or a slope of a line fit to data from a predetermined window. Further, the slope may be either positive or negative, depending on whether the physiological parameter is trending up or down. In one example, a patient may have an average parameter value that is in a normal range. For such patients, a slow up or down trend (e.g., a slope within a limit characterized by a slowly-changing parameter) may still yield measured values in the normal range. Accordingly, the slope may be considered in the context of a measured value. A gradual slope in the context of normal values may be associated with a low power mode. In addition to measured parameters, the trend may be calculated from characteristics of a plethysmographic waveform. For example, a trend or variability of a pulse amplitude may be used to determine if a high power mode or low power mode is appropriate. If the trend of the parameter counterindicates a low power mode, the user selection will be prohibited or delayed until the trend stabilizes and the low power mode is appropriate. In such embodiments, the display 20 may provide an indication that the high power mode is in effect.

Parameter calculations and trend data may be acquired and calculated according to any suitable technique. In an embodiment, a continuous wavelet processing system or processor (e.g., processor 48) may generate an SpO₂ signal or trend using a sensor signal. The SpO₂ signal or trend may, for example, be derived from a wavelet ratio surface, from a Lissajous figure, or both as discussed in U.S. Patent Publication No. 2006/0258921, which is hereby incorporated by reference herein in its entirety. Any other suitable technique for determining SpO₂ signals or trends may be used such as any suitable time domain techniques. In an embodiment, a wavelet processing system may process an SpO₂ signal or trend to determine if it is appropriate to trigger an alarm. For example, processor 48 in the system 10 may generate an SpO₂ signal and analyze the signal to determine when a patient's blood oxygenation levels are at dangerous levels and/or showing a dangerous pattern. In an embodiment, the system 10 may process an SpO₂ signal and determine a physiological parameter such that the high power mode is triggered if a moving average of the parameter is below a threshold and the instantaneous slope of the signal is non-positive (e.g., moving away from normal). Further, a threshold or range of slopes and parameter calculations associated with low power and high power modes may be determined based on empirical evidence or clinical data for individual parameters. In one embodiment, a sustained change in slope may trigger a switch to a high power mode.

In other embodiments of the present disclosure, a parameter variability may be used as an input to determining a switch between high power and low power modes. For example, the method 80 may use blood oxygen saturation variability or heart rate variability as an input. In one embodiment, a heart rate variability value greater than 50 milliseconds (ins) or 75 ins may associated with a high power mode. Determining variability of a parameter of interest may be accomplished by any suitable method, including a time domain methods. For example, the variability may be determined at least in part by calculating time domain statistics from the data collected from a pulse oximetry sensor, such as mean heart rate, standard deviation of pulse intervals (SDNN), square root of mean squared difference of successive pulse intervals (RMSSD), and the proportion of pulse intervals that differ from the mean (pNN50). In an alternative embodiment, parameter variability may be determined using frequency domain methods. A highly variable parameter may be associated with a high power mode while a relatively stable parameter may be associated with a low power mode. A threshold or range of variabilities associated with low power and high power modes may be determined based on empirical evidence or clinical data for individual parameters. For example, certain parameters may be associated with more natural variability for clinically normal patients.

In a specific embodiment, the switch between high power mode and low power mode may be based on alarm data calculated using a predetermined set of limits. For example, a pulse oximetry monitor such as those available from Nellcor Puritan Bennett LLC, may incorporate a SatSeconds™ alarm management system, such as the system disclosed in U.S. Pat. No. 5,865,736; U.S. Pat. No. 6,754,516; or U.S. Pat. No. 7,123,950, the disclosures of which are incorporated by reference in their entirety herein for all purposes. Generally speaking, SatSeconds™ alarm management operates by integrating an area between an alarm threshold and a patient's measured physiological parameters over time. For example, rather than sounding an alarm as soon as the patient's measured SpO₂ drops below a threshold value, the SatSeconds™ system measures an area by integrating the difference between a threshold SpO₂ and the patient's SpO₂ level when the patient's SpO₂ level is below the threshold. When the SatSeconds™ value exceeds a threshold value (e.g., a preset threshold or a user-input threshold), the caregiver may be alerted that the patient's oxygen saturation is too low. In one embodiment, when the SatSeconds™ value exceeds the threshold value, the monitor 14 may automatically switch to a high power mode from a low power mode.

Additionally, a monitor 14 or other medical device as provided herein may incorporate saturation pattern detection (SPD) alarms. In the flow diagram 116 shown in FIG. 6, SPD alarm limits may be used to determine if the monitor 14 is in the appropriate power mode. Based on blood oxygen saturation of a patient, calculated at step 120, the monitor 14 may determine if characteristic saturation patterns have occurred at step 122. Such patterns may use algorithm that perform a statistical method to find potential reciprocation peaks and nadirs in a trend of SpO₂ data. A nadir may be defined as a minimum SpO₂ value in a reciprocation. The peaks may include a rise peak (e.g., a maximum SpO₂ value in a reciprocation that occurs after the nadir) and/or a fall peak (e.g., a maximum SpO₂ value in a reciprocation that occurs before the nadir). An SPD index (SPDi) is created based upon these patterns and their severities, for example as provided in, U.S. Patent Publication Nos. 2006/0235324 to Lynn or 2010/0113909 to Batchelder et al., the specifications of both of which are incorporated by reference in their entirety herein for all purposes. In particular, the system 10 may calculate a physiological parameter from the data and determine if the calculated physiological parameter exceeds certain thresholds associated with alarm events. If the saturation pattern counter is below a threshold, the monitor 14 switches to a low power mode. Alternatively, the monitor 14 may allow a user to set the low power mode. If the saturation counter above an alarm threshold, the monitor 14 is automatically switched to high power mode at step 126 if not already in high power mode. Further, while the counter is above the threshold, the low power mode is prohibited.

While the disclosure may be susceptible to various modifications and alternative forms, specific embodiments have been shown by way of example in the drawings and will be described in detail herein. However, it should be understood that the disclosure is not intended to be limited to the particular forms disclosed. Rather, the disclosure is to cover all modifications, equivalents and alternatives falling within the spirit and scope of the disclosure as defined by the following appended claims. Further, it should be understood that elements of the disclosed embodiments may be combined or exchanged with one another.

What is claimed is:

1. A monitor, comprising:
an input circuit configured to receive a plethysmographic signal;
a memory storing an algorithm configured to calculate a physiological parameter and one or more characteristics of a trend of the physiological parameter based on the plethysmographic signal; and
a processor configured to execute the algorithm, wherein the processor is configured to execute the algorithm at a first rate per second associated with a high power mode or a second rate per second associated with a low power mode based on the one or more characteristics of the trend of the physiological parameter, wherein the first rate per second is higher than the second rate per second.

2. The monitor of claim 1, comprising a display, wherein the display is configured to display an indicator associated with the high power mode or the low power mode.

3. The monitor of claim 1, wherein the physiological parameter comprises an oxygen saturation.

4. The monitor of claim 1, wherein the one or more characteristics of the trend of the physiological parameter comprises a slope of an oxygen saturation, a pulse amplitude, a heart rate, or a combination thereof.

5. The monitor of claim 1, wherein the one or more characteristics of the trend of the physiological parameter comprises a slope, wherein an absolute value of the slope outside of a predetermined limit is associated with the high power mode.

6. The monitor of claim 1, wherein the one or more characteristics of the trend of the physiological parameter comprises a variability.

7. The monitor of claim 1, wherein the one or more characteristics of the trend of the physiological parameter comprises a number of alarm patterns detected.

8. The monitor of claim 7, wherein the low power mode is associated with a number of alarm patterns below a predetermined alarm limit.

9. A module configured to couple to a multi-parameter monitor, comprising:
an input circuit configured to receive a plethysmographic signal;
a memory storing an algorithm configured to calculate a trend of a physiological parameter based on the plethysmographic signal and determine if a low power mode or a high power mode is appropriate based on the trend;
a processor configured to execute the algorithm; and
a connector configured to couple the module to the multi-parameter monitor.

10. The module of claim 9, wherein an average power consumption of the module is less than 80 in mW.

11. The module of claim 9, wherein the low power mode comprises a reduction in processing power relative to the high power mode.

12. The module of claim 11, wherein the low power mode comprises a reduction in power distributed to one or more hardware components of the module.

13. The module of claim 9, wherein the low power mode comprises a reduction in calculation rate of the physiological parameter relative to the high power mode.

14. The module of claim 13, wherein the low power mode comprises a reduction in sampling rate of the plethysmographic signal.

15. The module of claim 9, wherein the low power mode is appropriate if an absolute value of a slope of the trend is within a predetermined limit.

16. The module of claim 9, wherein the low power mode is appropriate if an alarm index is below a predetermined alarm limit.

17. A system, comprising:
a sensor configured to generate a plethysmographic signal; and
a monitor coupled to the sensor, the monitor comprising:
an input circuit configured to receive the plethysmographic signal; and
a memory storing an algorithm configured to calculate a physiological parameter based on the plethysmographic signal, wherein the algorithm is configured to determine if the monitor can assume a low power mode or a high power mode based on one or more characteristics of a trend of the physiological parameter; and
a processor configured to execute the algorithm and switch between a first processing rate associated with the low power mode and a second processing rate associated with the high power mode.

18. The system of claim 17, wherein the one or more characteristics of the trend of the physiological parameter comprises a slope of an oxygen saturation, a pulse amplitude, a heart rate, or a combination thereof.

19. The system of claim 17, wherein the one or more characteristics of the trend of the physiological parameter comprises a slope, wherein an absolute value of the slope outside of a predetermined limit is associated with the high power mode.

20. The system of claim 17, wherein the one or more characteristics of the trend of the physiological parameter comprises a variability, wherein a variability outside of a predetermined limit is associated with the high power mode.
